(19) United States
(12) Patent Application Publication
(10) Pub. No.: US 2025/0005754 A1
(43) Pub. Date: Jan. 2, 2025

(54) AUTOMATIC GENERATION OF A REGION OF INTEREST IN A MEDICAL IMAGE FOR TUMOR TREATING FIELDS TREATMENT PLANNING

(71) Applicant: Novocure GmbH, Root D4 (CH)

(72) Inventors: Gil ZIGELMAN, Haifa (IL); Noa URMAN, Haifa (IL)

(73) Assignee: Novocure GmbH, Root D4 (CH)

(21) Appl. No.: 18/758,995

(22) Filed: Jun. 28, 2024

Related U.S. Application Data
(60) Provisional application No. 63/609,202, filed on Dec. 12, 2023.
(60) Provisional application No. 63/524,470, filed on Jun. 30, 2023.

(51) Int. Cl. G06T 7/00; G06T 7/11; G16H 20/40

(52) U.S. Cl. CPC G06T 7/0012; G06T 7/11; G16H 20/40; G06T 2207/30096

(57) ABSTRACT

A computer-implemented method for treatment planning for administering tumor treating fields to a subject, the method comprising: presenting on a display a slice through a medical image of the subject, wherein the medical image comprises voxels; and determining a segmentation of a minimum number of voxels of at least one tissue type in the slice of the medical image; receiving a user selection to automatically generate a region of interest (ROI) in the medical image for application of tumor treating fields to the subject; and automatically generating the region of interest in the medical image for application of tumor treating fields to the subject based on the segmentation of the minimum number of voxels of at least one tissue type in the slice of the medical image.

### CROSS-REFERENCE TO RELATED APPLICATIONS

This application claims priority to U.S. Provisional Application No. 63/609,202 filed Dec. 12, 2023 and U.S. Provisional Application No. 63/524,470 filed Jun. 30, 2023, the contents of each of which are incorporated by reference herein in their entirety. This application is related to U.S. patent application Ser. No. 18/750,582 filed Jun. 21, 2024, U.S. patent application Ser. No. 18/675,714 filed May 28, 2024, and U.S. Provisional Application No. 63/523,853 filed Jun. 28, 2023, the contents of each of which are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. In current commercial systems, TTFields are induced non-invasively into a region of interest by electrode assemblies (e.g., arrays of capacitively coupled electrodes, also called electrode arrays, transducer arrays or simply “transducers”) placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 is a flowchart depicting an example computer-implemented method for treatment planning for administering TTFields to a subject.

FIG. 2 is a flowchart depicting an example computer-implemented method for automatic generation of a ROI in a medical image of a subject.

FIG. 3 is a flowchart depicting an example computer-implemented method for generating transducer layouts for application of TTFields to a subject.

FIG. 4 depicts an example user interface of an application for manual segmentation of a medical image of a subject.

FIG. 5 depicts an example user interface of an application for automatic generation of a region of interest in a medical image of a subject.

FIG. 6 depicts an example user interface of a warning for automatic generation of a region of interest in a medical image of a subject.

FIGS. 7A, 7B, and 7C depict an example medical image of a subject at various stages of having a ROI automatically generated in the medical image.

FIG. 8 depicts an example system to apply alternating electric fields to a subject.

FIG. 9 depicts an example placement of transducers on a subject's head.

FIG. 10 depicts an example computer apparatus according to one or more embodiments described herein 60.

Various embodiments are described in detail below with reference to the accompanying drawings, where like reference numerals represent like elements.

### DESCRIPTION OF EMBODIMENTS

This application describes exemplary techniques for treatment planning for administering TTFields to a subject.

When treatment planning for administering TTFields to a subject, a region of interest (ROI) in the subject may need to be identified for determining a dosage of TTFields to be administered to the subject. Typically, identifying the ROI in a subject is performed manually by a user reviewing a medical image of the subject, where the medical image of the subject may have numerous slices and even more numerous voxels. As an example, the user may review each slice of the medical image of the subject and manually identify the ROI in the each slice. The ROI for the medical image may be the combination of the ROI's of each slice. This manual procedure requires a significant amount of time by the user, resulting in increased costs. Additionally, this manual procedure may cause delays in the TTFields treatment planning while a user is scheduled to review and manually process the medical image of the subject. Moreover, if a subject has several medical images to be used in the TTFields treatment planning, each medical image may need to be reviewed, and the ROI may need to be manually identified in each medical image by a user, which may cause even more time expended by the user, more costs associated therewith, and more delays in the TTFields treatment planning.

One or more embodiments described herein provide a technical solution to address this technical problem of identifying a ROI in a subject for determining a dosage of TTFields to be administered to the subject. In particular, the inventors discovered techniques for automatically generating a ROI in a medical image for TTFields treatment planning. With the inventive techniques, a user is no longer required to manually review a medical image or each slice a medical image to determine a ROI for the medical image. Instead, in some embodiments, the user need only segment a few voxels in the medical image, and a computer automatically generates a ROI for the medical image. With the inventive techniques, the amount of time required by a user to generate a ROI for a medical image may be significantly reduced compared to the typical manual procedures, which also may result in decreased costs for the inventive techniques compared to the typical manual procedures. Additionally, with the inventive techniques, delays in the TTFields treatment planning caused by needing to schedule a user to review and manually process the medical image of the subject may be significantly reduced due to the user not needing as much time to process a medical image using the inventive techniques. Moreover, if a subject has several medical images to be used in the TTFields treatment planning, the amount of time required by a user to generate a ROI for each medical image according to the inventive techniques may be significantly less than the amount of time required by a user employing the typical manual procedures, which may result in significant cost reductions associated therewith and less delays in the TTFields treatment planning, Due to the amount of data and the computational complexities involved, the technical solution cannot be performed by a human mind and, instead, needs to be performed by the computer-based techniques described herein.

The embodiments described herein further provide a practical application of generating transducer layouts for delivering TTFields treatment to a subject by avoiding the need to perform manual generation of a region of interest in a medical image of the subject. With the inventive techniques, the typical manual procedures for generating a region of interest in a medical image of a subject can be avoided, resulting in time savings by a user (e.g., a healthcare provider), reduced costs, and reduced delays in providing TTFields treatment planning. These and other technical improvements may be realized using the one or more embodiments described herein.

In some embodiments, the computer-based inventive techniques may include automatic generation of a ROI in a medical image for TTFields treatment planning. As an example, a user may first manually segment a minimum number of voxels in the medical image (e.g., 10 voxels) for at least one tissue type (e.g., tumor, resection, necrotic, etc.) and then select an “Auto ROI Generator” button in the user interface. In some embodiments, the user may also select a value for a margin (e.g., a proximal boundary zone (PBZ)) to be added to each segmented area of the medical image. Next, the computer may automatically determine the segmented areas for each tissue type based on the manually segmented voxels, add the margin to each segmented area to obtain expanded segmented areas, and combine the expanded segmented areas to obtain the region of interest for the medical image.

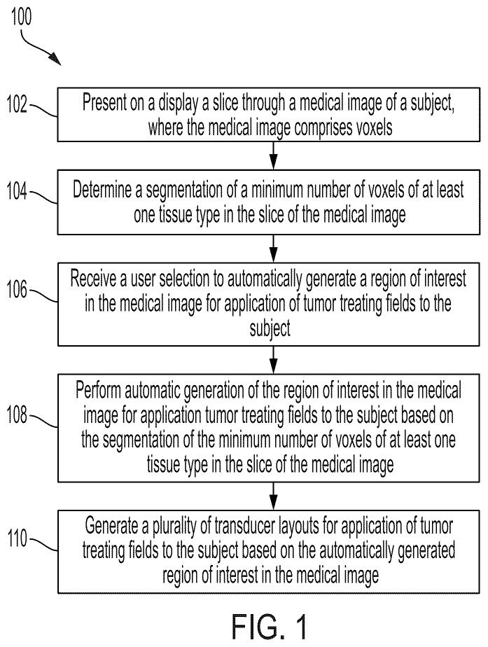

FIG. 1 depicts an example computer-implemented method 100 for treatment planning for administering TTFields to a subject. Certain steps of the method 100 are described as computer-implemented steps. The method 100 can be implemented by any suitable system or apparatus, such the apparatus 1000 of FIG. 10. While an order of operations is indicated in FIG. 1 for illustrative purposes, the timing and ordering of such operations may vary where appropriate without negating the purpose and advantages of the examples set forth in detail herein.

At step 102, the method 100 may include presenting on a display a slice through a medical image of the subject. In some embodiments, the medical image includes voxels and includes one or more slices. The medical image may include at least one of computed tomography (CT) medical image, a magnetic resonance imaging (MRI) medical image, or a positron emission tomography (PET) medical image. In some embodiments, the medical image of the subject may include an image of the torso and/or the head of the subject.

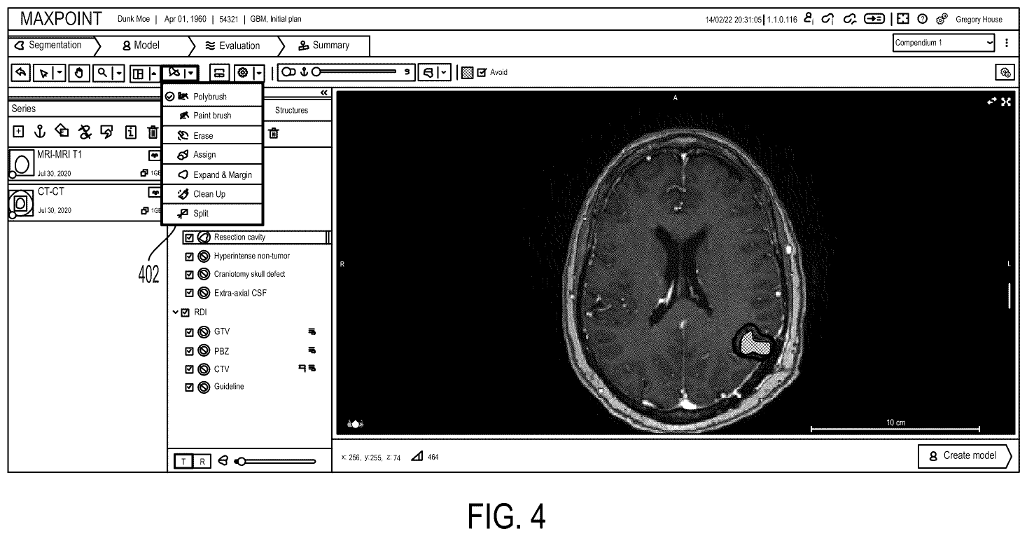

At step 104, the method 100 may include determining a segmentation of a minimum number of voxels of at least one tissue type in the slice of the medical image. The at least one tissue type may include at least one of a tumor, a gross tumor volume (GTV), a resection cavity, a necrotic area, or an enhancing tumor. A GTV may denote a macroscopic tumor volume, which may be used as a central target volume for treatment. A resection cavity may refer to a cavity caused by removal of a tissue, a structure, or an organ. A necrotic area may refer to dead cells through injury or diseases. An enhancing tumor may refer to a remaining tumor area injected with contrast material so that such area becomes easier to be detected in a medical image, for example, in a MRI medical image. In some embodiments, a user interface may be provided to the user for determining a segmentation of at least one tissue type in the slice of the medical image. As an example, FIG. 4 depicts an example user interface of an application for manual segmentation of a medical image of a subject.

In some embodiments, step 104 may be based on user input. For example, a user may select voxels in a slice of a medical image as a particular tissue type, and these user-selected voxels may be identified as the determined segmentation of a minimum number of voxels of at least one tissue type in the slice of the medical image. As an example, the minimum number of voxels of the medical image for segmentation may be between 5 voxels and 25 voxels. As an example, the minimum number of voxels of the medical image for segmentation may be 10 voxels. In some embodiments, if the minimum number of voxels of the medical image for segmentation is not satisfied, a warning may be provided to the user.

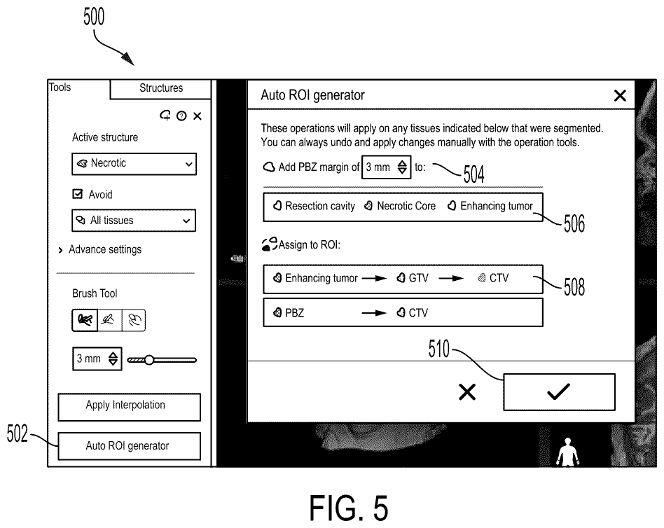

At step 106, the method 100 may include receiving a user selection to automatically generate a ROI in the medical image for application of the TTFields to the subject. In some embodiments, the user may select a button in a user interface, such as an “Auto ROI Generator” button in the user interface (for example, “Auto ROI Generator” button 502 in FIG. 5), and this selection may be received to then automatically generate a ROI in the medical image. In some embodiments, the user may also input a margin (e.g., a proximal boundary zone (PBZ)) to add to a segmented area as part of the automatic generation of the ROI.

At step 108, the method 100 may include performing automatic generation of the ROI in the medical image for application of the TTFields to the subject based on the segmentation of the minimum number of voxels of at least one tissue type in the slice of the medical image from step 104. In some embodiments, a segmented area in the medical image may be automatically generated based on the segmentation of the minimum number of voxels of at least one tissue type from step 104. In some embodiments, the automatic segmentation may be performed using the techniques discussed in commonly-owned U.S. Patent Application Publication No. 2021/0201572, entitled “METHODS, SYSTEMS, AND APPARATUSES FOR IMAGE SEGMENTATION,” the contents of which are incorporated by reference herein in their entirety. In some embodiments, the ROI may include a clinical tumor volume (CTV). A CTV may refer to a tissue volume that contains a gross tumor volume (GTV) and subclinical microscopic malignant lesions. In some embodiments, generating the ROI in the medical image may include adding a proximal boundary zone (PBZ) to the GTV to obtain the CTV in the medical image. In some embodiments, a user interface may be provided to the user for initiating automatic generation of the ROI. As an example, FIG. 5 depicts an example user interface of an application for automatic generation of a ROI in a medical image of a subject.

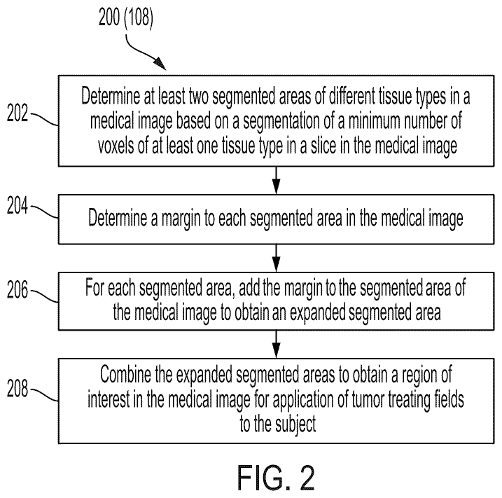

In some embodiments, the automatic generation of the ROI at step 108 may include determining a segmented area of a tissue type in the medical image based on the segmentation of the minimum number of voxels of at least one tissue type in the slice in the medical image, determining the PBZ for the segmented area of the medical image, and then adding the PBZ to the segmented area of the medical image to obtain the ROI in the medical image for application of the TTFields to the subject. In particular, the segmented area may be a tumor, a GTV, a resection cavity, a necrotic area, an enhancing tumor, or a non-enhancing tumor. As used herein, the phrase “segmented area” may refer to a three-dimensional volume in a medical image. In some embodiments, step 106 may be implemented using method 200 in FIG. 2, which is discussed further below.

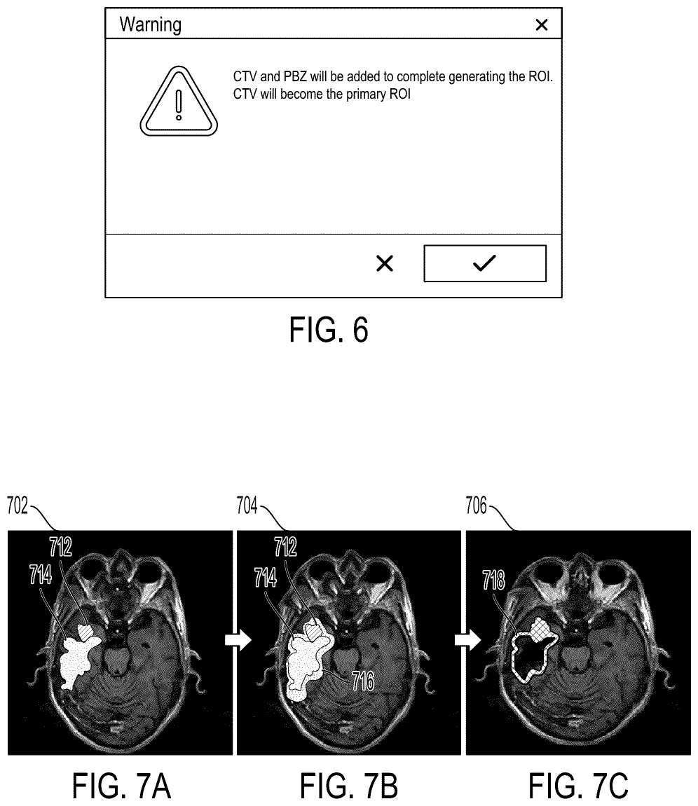

In some embodiments, during automatically generating the ROI, a calculation warning may be provided via a user interface. In some embodiments, a user interface may be provided to the user for providing a warning for automatic generation of the ROI. As an example, FIG. 6 depicts an example user interface of a warning for automatic generation of a ROI in a medical image of a subject. In some embodiments, the warning may indicate that a minimum number of voxels were not selected (e.g., in step 104) and, as such, automatic generation of the ROI cannot proceed. In some embodiments, the warning may indicate at least one of the following: the ROI in the medical image does not contain a minimum number of gray and/or white matter voxels, the ROI in the medical image does not contain a minimum number of enhancing tumor voxels, only regions of interest having gray and/or white matter voxels or enhancing tumor voxels will be presented, and/or the like including combinations and/or multiples thereof. If one or more of these warnings is received, the computer system may provide the user with an opportunity to resolve the problem. For example, to address the warning that the ROI in the medical image does not contain a minimum number of gray and/or white matter voxels is presented, the user may need to revise the size of the segmentation in step 104 so as to include additional gray and/or white matter voxels. For example, to address the warning that the ROI in the medical image does not contain a minimum number of enhancing tumor voxels, the user may need to revise the size of the segmentation in step 104 so as to include additional enhancing tumor voxels. For example, to address the warning that only regions of interest having gray and/or white matter voxels or enhancing tumor voxels will be presented, the user may need to revise the size of the segmentation in step 104 so as that voxels not in the ROI will be presented.

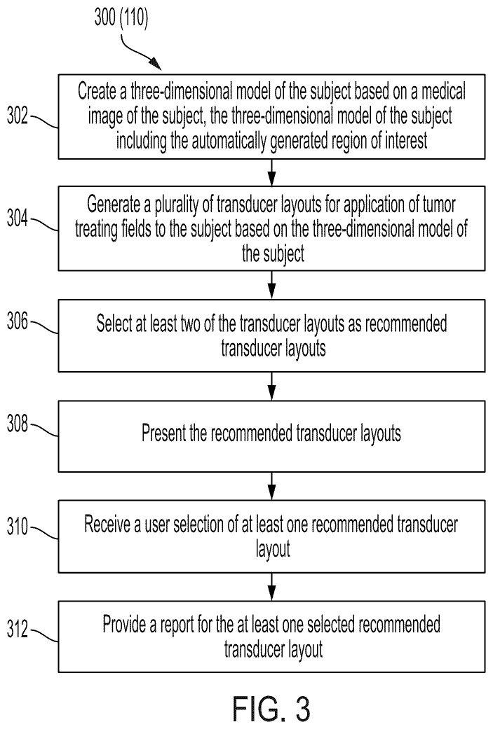

At step 110, the method 100 may include generating a plurality of transducer layouts for application of the TTFields to the subject based on the automatically generated ROI in the medical image. In some embodiments, at least one of the transducer layouts may include two pairs of transducers for placement on the subject at transducer locations. In some embodiments, at least one of the transducer layouts may include one pair of transducers for placement on the subject at transducer locations. In some embodiments, the transducer layouts may be generated based on at least one of a three-dimensional model of the subject, a three-dimensional model of a generic subject, or geometric calculations of the subject. In some embodiments, step 110 may be implemented using method 300 in FIG. 3, which is discussed further below.

FIG. 2 illustrates a flowchart depicting an example method 200 for automatic generation of a ROI in a medical image of a subject. The method 200 may be used to implement step 108 of FIG. 1. Certain steps of the method 200 may be described as computer-implemented steps. The method 200 may be implemented by any suitable system or apparatus, such as the apparatus 1000 of FIG. 10. While an order of operations is indicated in FIG. 2 for illustrative purposes, the timing and ordering of such operations may vary where appropriate without negating the purpose and advantages of the examples set forth in detail herein.

At step 202, the method 200 may include determining at least two segmented areas of different tissue types in a medical image based on a segmentation of a minimum number of voxels of at least one tissue type in the slice in the medical image. As an example, the at least two segmented areas may include at least two of a tumor, a gross tumor volume, a resection cavity, a necrotic area, or an enhancing tumor, or a non-enhancing tumor. The segmentation of the medical image with the minimum number of voxels may be selected by a user in step 104.

At step 204, the method 200 may include determining a margin for each segmented area in the medical image. The margin may be used to expand the segmented area, which may be useful if there is uncertainty as to where the segmentation in the subject actually lies (e.g., voxels on the edge or separating tumor tissue from non-tumor tissue). In some embodiments, the margin may be a PBZ for each segmented area in the medial image. In some embodiments, the margin (or PBZ) may be a same or a different value for each segmented area in the medical image. In some embodiments, the margin (or PBZ) may be a user-selectable value for the segmented areas in the medical image. In some embodiments, the margin may be a distance measurement. As an example, the margin may be between 1 mm and 15 mm, or between 1 mm and 20 mm. As an example, the margin may be 3 mm. In some embodiments, the margin may be a percentage of a size of the segmented area. As an example, the margin may be between 0.1% to 5.0%, or between 0.01% and 10.0%, of a size of the segmented area. As an example, the margin may be 0.01%, 0.1%, 0.5%, or 1.0%.

At step 206, the method 200 may include, for each segmented area, adding the margin to the segmented area of the medical image to obtain an expanded segmented area. In some embodiments, each expanded segmented area corresponds to a segmented area.

At step 208, the method 200 may include combining the expanded segmented areas to obtain the ROI in the medical image for application of the TTFields to the subject. The ROI may include overlapping and non-overlapping portions of the expanded segmented areas.

In some embodiments, the order of steps 206 and 208 may be reversed. As an example, the segmented areas may first be combined into a combined segmented area and then a margin can be added to the combined segmented area to obtain the ROI in the medical image.

As an example of implementing aspects of method 200, FIGS. 7A-7C depict an example medical image of a subject at various stages of having a ROI automatically generated in the medical image.

FIG. 3 illustrates a flowchart depicting an example method 300 for generating transducer layouts for application of TTFields to a subject. The method 300 may be used to implement step 110 of FIG. 1. Certain steps of the method 300 may be described as computer-implemented steps. The method 300 may be implemented by any suitable system or apparatus, such as the apparatus 1000 of FIG. 10. While an order of operations is indicated in FIG. 3 for illustrative purposes, the timing and ordering of such operations may vary where appropriate without negating the purpose and advantages of the examples set forth in detail herein.

At step 302, the method 300 may include creating a three-dimensional (3D) model of the subject based on the medical image, where the 3D model of the subject includes the automatically generated ROI. In some embodiments, the 3D model may include a 3D conductivity map. The 3D conductivity map may depict electrical conductivity of body tissues of the subject. In some embodiments, creating the 3D model may include performing calculations to determine conductivity of tissues of the subject based on the medical image and the tissue types in the medical image. As one example, creating the 3D model may include assigning tissue types and associated conductivities to voxels of the 3D model of the subject. In some embodiments, creating the 3D model of the subject may include automatically segmenting normal tissue in the medical image. In some embodiments, after the 3D model of the subject is created, the method 300 can include receiving user approval of a 3D conductivity map associated with the 3D model. In some embodiments, the auto-segmented normal tissues, such as gray and white matter, skull, scalp, and cerebrospinal fluid (CSF), may be automatically added to the 3D model. In some embodiments, creating the 3D model of the subject at step 302 can be performed using techniques in commonly-owned U.S. Patent Application Publication No. 2021/0201572, entitled “METHODS, SYSTEMS, AND APPARATUSES FOR IMAGE SEGMENTATION”, the contents of which are incorporated by reference herein in their entirety.

At step 304, the method 300 may include generating a plurality of transducer layouts for application of TTFields to the subject based on the 3D model of the subject. The transducer layouts may define one or more locations, relative to the subject, for placing transducers. In some embodiments, the plurality of the transducer layouts may include four locations on the subject to place four respective transducers, such as on a head or torso of the subject. In some embodiments, the plurality of the transducer layouts may include two locations on the subject to place two respective transducers, such as on a head or torso of the subject. In some embodiments, each of the transducers may include one or more electrode elements. The electrode elements may be any suitable type or material. For example, at least one electrode element may include a ceramic dielectric layer, a polymer film, and/or the like including combinations and/or multiples thereof. Generating the plurality of transducer layouts may be performed after receiving a selection by a user from a user interface to begin the generating. In some embodiments, generating the plurality of transducer layouts at step 304 can be performed using techniques in commonly-owned U.S. Patent Application Publication No. 2021/0201572, entitled “METHODS, SYSTEMS, AND APPARATUSES FOR IMAGE SEGMENTATION”, the contents of which are incorporated by reference herein in their entirety.

At step 306, the method 300 may include selecting at least two of the plurality of transducer layouts as recommended transducer layouts for presentation to the user. In some embodiments, at least one of the recommended transducer layouts may have a highest dose of TTFields delivered to the ROI, delivered a tumor progression area, and/or the like including combinations and/or multiples thereof. In some embodiments, at least one of the recommended transducer layouts may be a transducer layout that is in a shifted or rotated position compared to a transducer layout having a highest dose of TTFields delivered to the ROI. In some embodiments, at least three of the recommended transducer layouts may have three highest doses of TTFields delivered to the ROI.

At step 308, the method 300 may include presenting the recommended transducer array layouts. For example, the method 300 may include presenting at least four recommended transducer layouts, although more or fewer transducer layouts may be presented in other examples. In some embodiments, presenting the recommended transducer layouts may include presenting information on the recommended transducer layouts via a user interface. The information may include one or more of the following: doses of TTFields delivered to the ROI for each of the recommended transducer layout, a medical image slice overlaid with a dose of TTFields for at least one recommended transducer layout, a two-dimensional graph comparing percentage volume of ROI and percentage dose of TTFields for at least one recommended transducer layout, an image of the subject depicting locations of electrode elements for at least one recommended transducer layout, a two-dimensional graph depicting a cumulative dose of TTFields across the ROI for at least one recommended transducer layout, a two-dimensional graph depicting a dose of TTFields across the ROI for at least one recommended transducer layout, a percentage of overlap between electrode elements of two recommended transducer layouts, a percentage of overlap between adhesive portions of two recommended transducer layouts, and/or the like including combinations and/or multiples thereof.

At step 310, the method 300 may include receiving a user selection of at least one recommended transducer array layout. In some embodiments, the user may select a primary transducer layout and an alternative transducer layout. To make the selection, the user may accept a primary layout as a first layout then review and evaluate alterative layouts and select an alternative layout as a second layout. For example, the user may select one (e.g., a primary) of the transducer layouts for use during a first period of time. The user may select another (e.g., an alternate) of the transducer layouts for use during a second period of time after the first period of time.

At step 312, the method 300 may include provide a report for the at least one selected recommended transducer layout. In some embodiments, the report may depict locations of transducers of the selected recommended transducer layout on the subject in a plurality of views. In some embodiments, the report may provide dosages of the TTFields applied to the subject. It should be appreciated that different reports may be provided, for example, depending on anticipated target of the report (e.g., a first report type for the subject, a second report type for inclusion in the subject's medical records).

Turning to FIG. 4, FIG. 4 depicts an example user interface of an application for manual segmentation of a medical image of a subject. Specifically, FIG. 4 shows multiple user-selectable options 402 (e.g., user-selectable icons) to manually segment a slice presented in a drop down menu. Examples of the user-selectable options 402 may include, for example, a user-selectable icon to autofill a region (e.g., a polybrush option), a user-selectable icon to select the region without autofill (e.g., a paint brush option), a user-selectable icon to erase a segmentation (e.g., an erase option), a user-selectable icon to assign a tissue type (e.g., an assign option), a user-selectable icon to expand a border of the region (e.g., an expand & margin option), a user-selectable icon to clean up a segmented slice, a user-selectable icon to split a segmentation, and/or the like including combinations and/or multiples thereof. In some embodiments, the user-selectable options 402 provide a user with a set of tools to select. The tools may be used for segmenting abnormal tissues. For example, a polybrush or paint brush may be used to outline an abnormal tissue region. The abnormal tissue may be any undesirable type of tissue, such as a tumor, necrotic tissue, a prior surgical area (e.g., a resection cavity), and/or the like including combinations and/or multiples thereof.

FIG. 5 depicts an example user interface of an application for automatic generation of a ROI in a medical image of a subject. As the example shown in FIG. 5, a user may start automatic generation of the ROI by clicking an “Auto ROI Generator” button at 502, and an “Auto ROI Generator” window would show up. The window may display at 504 a user-adjustable value for a PBZ margin to be added to and/or for expanding a segmented area in the medical image to obtain the expanded segmented area in the medical image. The window may display at 506 a user-selectable tissue type for the segmented area to add the PBZ margin. Examples of the user-selectable tissue type may include resection cavity, necrotic core, or enhancing tumor. The window may display at 508 an indication of how the margin will be combined with the different tissue types. As an example, the enhancing tumor may be assigned to a GTV, which may be assigned to a CTV in the ROI. As an example, the PBZ may be assigned to a CTV in the ROI. Upon making these selections, the user may click the button at 510 to accept these parameters and begin the automatic generation of the ROI using the selected parameters.

FIG. 6 depicts an example user interface of a warning for automatic generation of a ROI in a medical image of a subject. FIG. 6 shows an example where if the minimum number of voxels of the medical image for segmentation is not satisfied, a warning is provided to a user. As an example, the warning in FIG. 6 may indicates that CTV and PBZ will be added to complete generating the ROI and the CTV will become a primary ROI.

FIGS. 7A-7C depict an example medical image of a subject at various stages of having a ROI automatically generated in the medical image according to the method 200 in FIG. 2. In FIG. 7A, at step 202, two segmented areas are generated in the medical image, namely an enhancing tumor segmented area 712 and a resection cavity segmented area 714. In FIG. 7B, at step 206, a PBZ margin 716 (e.g., 3 mm) is added to the enhancing tumor segmented area 712 and is also added to the resection cavity segmented area 714, resulting in enhanced segmented areas. In FIG. 7C, at step 208, the two enhanced segmented areas are combined to obtain the ROI 718.

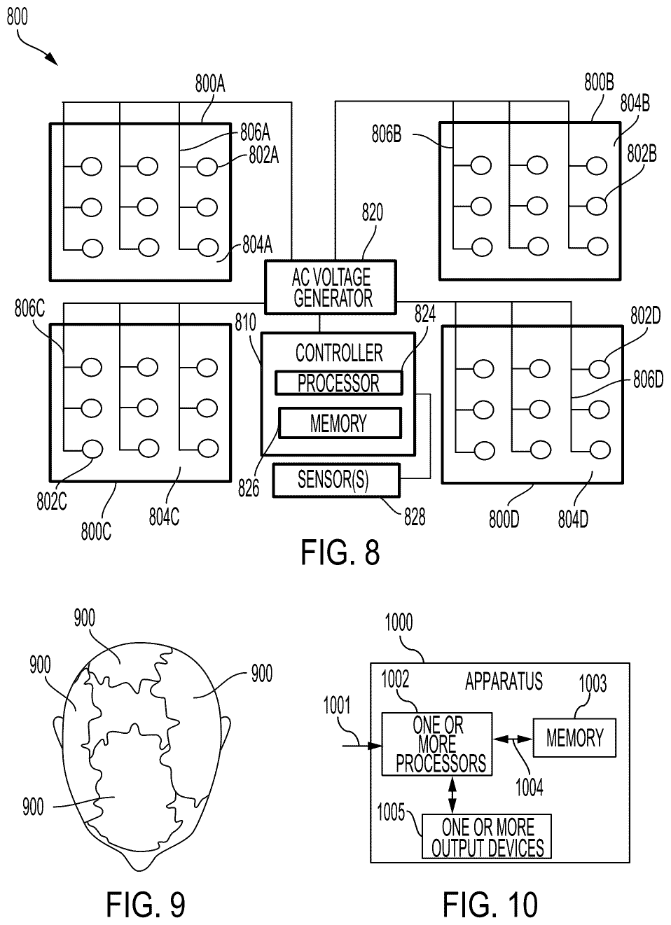

FIG. 8 depicts an example system 800 to apply alternating electric fields (e.g., TTFields) to the subject's body. The system may be used for treating a target region of a subject's body with an alternating electric field. In an example, the target region may be in the subject's brain, and an alternating electric field may be delivered to the subject's body via two pairs of transducer arrays positioned on a head of the subject's body (such as, for example, in FIG. 9, which has four transducers 900). In an example, the target region may be in the subject's torso, and an alternating electric field may be delivered to the subject's body via two pairs of transducer arrays positioned on at least one of a thorax, an abdomen, or one or both thighs of the subject's body. Other transducer array placements on the subject's body may be possible.

The example apparatus 800 has four transducers (or “transducer arrays”) 800A-D. Each transducer 800A-D may include substantially flat electrode elements 802A-D positioned on a substrate 804A-D and electrically and physically connected (e.g., through conductive wiring 806A-D). The substrates 804A-D may include, for example, cloth, foam, flexible plastic, and/or conductive medical gel. Two transducers (e.g., 800A and 800D) may be a first pair of transducers configured to apply an alternating electric field to a target region of the subject's body. The other two transducers (e.g., 800B and 800C) may be a second pair of transducers configured to similarly apply an alternating electric field to the target region.

The transducers 800A-D may be coupled to an AC voltage generator 820, and the system may further include a controller 810 communicatively coupled to the AC voltage generator 820. The controller 810 may include a computer having one or more processors 824 and memory 826 accessible by the one or more processors. The memory 826 may store instructions that when executed by the one or more processors control the AC voltage generator 820 to induce alternating electric fields between pairs of the transducers 800A-D according to one or more voltage waveforms and/or cause the computer to perform one or more methods disclosed herein. The controller 810 may monitor operations performed by the AC voltage generator 820 (e.g., via the processor(s) 824). One or more sensor(s) 828 may be coupled to the controller 810 for providing measurement values or other information to the controller 810.

The electrode elements 802A-D may be capacitively coupled. In one example, the electrode elements 802A-D may be ceramic electrode elements coupled to each other via conductive wiring 806A-D. When viewed in a direction perpendicular to its face, the ceramic electrode elements may be circular shaped or non-circular shaped. In other embodiments, the array of electrode elements may not be capacitively coupled, and there may be no dielectric material (such as ceramic, or high dielectric polymer layer) associated with the electrode elements.

The structure of the transducers 800A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one or more electrode elements). For example, the transducer may include one, two, three, four, five, six, seven, eight, nine, ten, or more electrode elements (e.g., twenty electrode elements). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. The transducer may be conductive or non-conductive. In some embodiments, an AC signal may be capacitively coupled into the subject's body. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

FIG. 10 depicts an example computer apparatus for use with the embodiments herein. As an example, the apparatus 1000 may be a computer to implement certain inventive techniques disclosed herein, such as selecting transducer locations for delivering TTFields to a subject. As one example, method 100 of FIG. 1 may be performed by a computer, such as apparatus 1000. As another example, method 200 of FIG. 2 may be performed by a computer, such as the apparatus 1000, which may the same computer or a different computer than the computer used to perform method 100 of FIG. 1. As another example, method 300 of FIG. 3 may also be performed by a computer, such as the apparatus 1000, which may the same computer or a different computer than the computer used to perform methods 100 and 200. In some embodiments, controller 810 of FIG. 8 may be implemented with apparatus 1000. The apparatus 1000 may include one or more processors 1002, memory 1003, one or more input devices (not shown), and one or more output devices 1005.

In some embodiments, based on input 1001, the one or more processors 1002 may generate control signals to control the voltage generator. As an example, input 1001 is user input. As an example, input 1001 may be from another computer in communication with the apparatus 1000. The input 1001 may be received in conjunction with one or more input devices (not shown) of the apparatus 1000.

The memory 1003 may be accessible by the one or more processors 1002 (e.g., via a link 1004) so that the one or more processors 1002 can read information from and write information to the memory 1003. The memory 1003 may store instructions that, when executed by the one or more processors 1002, implement one or more embodiments described herein.

The one or more output devices 1005 may provide the status of the operation of the invention, such as transducer array selection, voltages being generated, and other operational information. The output device(s) 1005 may provide visualization data according to some embodiments described herein.

The apparatus 1000 may be an apparatus for treatment planning for administering tumor treating fields to a subject, the apparatus including: one or more processors (such as one or more processors 1002); and memory (such as memory 1003) accessible by the one or more processors, the memory storing instructions that when executed by the one or more processors, cause the apparatus to perform one or more methods described herein.

The memory 1004 may be a non-transitory processor readable medium containing a set of instructions thereon for treatment planning for administering tumor treating fields to a subject, wherein when executed by a processor (such as processor 1002), the instructions cause the processor to perform one or more methods described herein.

### ILLUSTRATIVE EMBODIMENTS

The invention includes other illustrative embodiments (“Embodiments”) as follows.

Embodiment 1. A computer-implemented method for treatment planning for administering tumor treating fields to a subject, the method comprising: presenting on a display a slice through a medical image of the subject, wherein the medical image comprises voxels; and determining a segmentation of a minimum number of voxels of at least one tissue type in the slice of the medical image; receiving a user selection to automatically generate a region of interest (ROI) in the medical image for application of tumor treating fields to the subject; and automatically generating the region of interest in the medical image for application of tumor treating fields to the subject based on the segmentation of the minimum number of voxels of at least one tissue type in the slice of the medical image.

Embodiment 2: The computer-implemented method of Embodiment 1, wherein determining the segmentation of a minimum number of voxels of at least one tissue type in the slice of the medical image comprises: determining a segmentation of a minimum number of voxels of a tumor in the medical image.

Embodiment 3: The computer-implemented method of Embodiment 1, wherein determining the segmentation of a minimum number of voxels of at least one tissue type in the slice of the medical image comprises: determining a segmentation of a minimum number of voxels of a gross tumor volume in the medical image.

Embodiment 4: The computer-implemented method of Embodiment 1, wherein determining the segmentation of a minimum number of voxels of at least one tissue type in the slice of the medical image comprises at least one of: determining a segmentation of a minimum number of voxels of a resection cavity in the medical image; determining a segmentation of a minimum number of voxels of a necrotic area in the medical image; or determining a segmentation of a minimum number of voxels of an enhancing tumor in the medical image.

Embodiment 5: The computer-implemented method of Embodiment 1, wherein the region of interest in the medical image for application of tumor treating fields to the subject comprises a clinical tumor volume in the medical image for application of tumor treating fields to the subject.

Embodiment 6: The computer-implemented method of Embodiment 1, wherein automatically generating the region of interest in the medical image for application of tumor treating fields to the subject comprises: adding a proximal boundary zone (PBZ) to a gross tumor volume (GTV) to obtain a clinical tumor volume (CTV) in the medical image for application of tumor treating fields to the subject.

Embodiment 7: The computer-implemented method of Embodiment 1, wherein automatically generating the region of interest in the medical image for application of tumor treating fields to the subject comprises: determining a segmented area of a tissue type in the medical image based on the segmentation of the minimum number of voxels of at least one tissue type in the slice in the medical image; determining a proximal boundary zone (PBZ) for the segmented area of the medical image; and adding the PBZ to the segmented area of the medical image to obtain the region of interest in the medical image for application of tumor treating fields to the subject.

Embodiment 8: The computer-implemented method of Embodiment 7, wherein the segmented area is a tumor, a gross tumor volume, a resection cavity, a necrotic area, an enhancing tumor, or a non-enhancing tumor.

Embodiment 9: The computer-implemented method of Embodiment 1, wherein automatically generating the region of interest in the medical image for application of tumor treating fields to the subject comprises: determining at least two segmented areas of different tissue types in the medical image based on the segmentation of the minimum number of voxels of at least one tissue type in the slice in the medical image; determining a margin for each segmented area in the medical image; adding the margin to each segmented area of the medical image to obtain expanded segmented areas; and combining the expanded segmented areas to obtain the region of interest in the medical image for application of tumor treating fields to the subject.

Embodiment 10: The computer-implemented method of Embodiment 9, wherein the at least two segmented areas include at least two of a tumor, a gross tumor volume, a resection cavity, a necrotic area, an enhancing tumor, or a non-enhancing tumor.

Embodiment 10A: The computer-implemented method of Embodiment 9, wherein the region of interest comprises overlapping and non-overlapping portions of the expanded segmented areas.

Embodiment 10B: The computer-implemented method of Embodiment 9, wherein the PBZ is a same value for each segmented area in the medical image.

Embodiment 10C: The computer-implemented method of Embodiment 9, wherein the PBZ is a different value for each segmented area in the medical image.

Embodiment 10D: The computer-implemented method of Embodiment 9, wherein the PBZ is a user-selectable value for the segmented areas in the medical image.

Embodiment 11: The computer-implemented method of Embodiment 1, wherein automatically generating the region of interest in the medical image for application of tumor treating fields to the subject comprises: determining a proximal boundary zone (PBZ) for expanding a segmented area in the medical image to obtain an expanded segmented area in the medical image.

Embodiment 12: The computer-implemented method of Embodiment 15, wherein the PBZ for the segmented area of the medical image is 3 mm.

Embodiment 12A: The computer-implemented method of Embodiment 15, wherein the PBZ for the segmented area of the medical image is between 1 mm and 15 mm or between 1 mm and 20 mm.

Embodiment 13: The computer-implemented method of Embodiment 1, wherein automatically generating the region of interest in the medical image for application of tumor treating fields to the subject comprises: providing for display a user-adjustable value for a proximal boundary zone (PBZ) for expanding a segmented area in the medical image to obtain an expanded segmented area in the medical image.

Embodiment 14: The computer-implemented method of Embodiment 1, wherein the minimum number of voxels of the medical image for segmentation is 10 voxels.

Embodiment 14A: The computer-implemented method of Embodiment 1, wherein the minimum number of voxels of the medical image for segmentation is between 5 voxels and 25 voxels.

Embodiment 15: The computer-implemented method of Embodiment 1, wherein if the minimum number of voxels of the medical image for segmentation is not satisfied, a warning is provided to a user.

Embodiment 16: The computer-implemented method of Embodiment 1, further comprising: generating a plurality of transducer layouts for application of tumor treating fields to the subject based on the automatically generated region of interest in the medical image.

Embodiment 17: The computer-implemented method of Embodiment 1, further comprising: creating a three-dimensional model of the subject based on the medical image, the three-dimensional model of the subject including the automatically generated region of interest; generating a plurality of transducer layouts for application of tumor treating fields to the subject based on the three-dimensional model of the subject; selecting at least two of the plurality of transducer layouts as recommended transducer layouts; presenting the recommended transducer layouts; receiving a user selection of at least one recommended transducer layout; and providing a report for the at least one selected recommended transducer layout.

Embodiment 18: The computer-implemented method of Embodiment 1, wherein the medical image comprises at least one of a computed tomography (CT) medical image, a magnetic resonance imaging (MRI) medical image, or a positron emission tomography (PET) medical image.

Embodiment 18A: The computer-implemented method of Embodiment 1, wherein the medical image comprises a torso of the subject.

Embodiment 18B: The computer-implemented method of Embodiment 1, wherein the medical image comprises a head of the subject.

Embodiment 19: A non-transitory processor readable medium containing a set of instructions thereon for treatment planning for administering tumor treating fields to a subject, wherein when executed by a processor, the instructions cause the processor to perform a method comprising: presenting on a display a slice through a medical image of the subject, wherein the medical image comprises voxels; and determining a segmentation of a minimum number of voxels of at least one tissue type in the slice of the medical image; receiving a user selection to automatically generate a region of interest (ROI) in the medical image for application of tumor treating fields to the subject; and automatically generating the region of interest in the medical image for application of tumor treating fields to the subject based on the segmentation of the minimum number of voxels of at least one tissue type in the slice of the medical image.

Embodiment 20: An apparatus for treatment planning for administering tumor treating fields to a subject, the apparatus comprising: one or more processors; and memory accessible by the one or more processors, the memory storing instructions that when executed by the one or more processors, cause the apparatus to perform a method comprising: presenting on a display a slice through a medical image of the subject, wherein the medical image comprises voxels; and determining a segmentation of a minimum number of voxels of at least one tissue type in the slice of the medical image; receiving a user selection to automatically generate a region of interest (ROI) in the medical image for application of tumor treating fields to the subject; and automatically generating the region of interest in the medical image for application of tumor treating fields to the subject based on the segmentation of the minimum number of voxels of at least one tissue type in the slice of the medical image.

Embodiment 21: A method, machine, manufacture, and/or system substantially as shown and described.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A computer-implemented method for treatment planning for administering tumor treating fields to a subject, the method comprising:
presenting on a display a slice through a medical image of the subject, wherein the medical image comprises voxels; and
determining a segmentation of a minimum number of voxels of at least one tissue type in the slice of the medical image;
receiving a user selection to automatically generate a region of interest (ROI) in the medical image for application of tumor treating fields to the subject; and
automatically generating the region of interest in the medical image for application of tumor treating fields to the subject based on the segmentation of the minimum number of voxels of at least one tissue type in the slice of the medical image.

2. The method of claim 1, wherein determining the segmentation of a minimum number of voxels of at least one tissue type in the slice of the medical image comprises:
determining a segmentation of a minimum number of voxels of a tumor in the medical image.

3. The method of claim 1, wherein determining the segmentation of a minimum number of voxels of at least one tissue type in the slice of the medical image comprises:
determining a segmentation of a minimum number of voxels of a gross tumor volume in the medical image.

4. The method of claim 1, wherein determining the segmentation of a minimum number of voxels of at least one tissue type in the slice of the medical image comprises at least one of:
determining a segmentation of a minimum number of voxels of a resection cavity in the medical image;
determining a segmentation of a minimum number of voxels of a necrotic area in the medical image; or
determining a segmentation of a minimum number of voxels of an enhancing tumor in the medical image.

5. The method of claim 1, wherein the region of interest in the medical image for application of tumor treating fields to the subject comprises a clinical tumor volume in the medical image for application of tumor treating fields to the subject.

6. The method of claim 1, wherein automatically generating the region of interest in the medical image for application of tumor treating fields to the subject comprises:
adding a proximal boundary zone (PBZ) to a gross tumor volume (GTV) to obtain a clinical tumor volume (CTV) in the medical image for application of tumor treating fields to the subject.

7. The method of claim 1, wherein automatically generating the region of interest in the medical image for application of tumor treating fields to the subject comprises:
determining a segmented area of a tissue type in the medical image based on the segmentation of the minimum number of voxels of at least one tissue type in the slice in the medical image;
determining a proximal boundary zone (PBZ) for the segmented area of the medical image; and
adding the PBZ to the segmented area of the medical image to obtain the region of interest in the medical image for application of tumor treating fields to the subject.

8. The method of claim 7, wherein the segmented area is a tumor, a gross tumor volume, a resection cavity, a necrotic area, an enhancing tumor, or a non-enhancing tumor.

9. The method of claim 1, wherein automatically generating the region of interest in the medical image for application of tumor treating fields to the subject comprises:
determining at least two segmented areas of different tissue types in the medical image based on the segmentation of the minimum number of voxels of at least one tissue type in the slice in the medical image;
determining a margin for each segmented area in the medical image;
adding the margin to each segmented area of the medical image to obtain expanded segmented areas; and
combining the expanded segmented areas to obtain the region of interest in the medical image for application of tumor treating fields to the subject.

10. The method of claim 9, wherein the at least two segmented areas include at least two of a tumor, a gross tumor volume, a resection cavity, a necrotic area, an enhancing tumor, or a non-enhancing tumor.

11. The method of claim 1, wherein automatically generating the region of interest in the medical image for application of tumor treating fields to the subject comprises:
determining a proximal boundary zone (PBZ) for expanding a segmented area in the medical image to obtain an expanded segmented area in the medical image.

12. The method of claim 11, wherein the PBZ for the segmented area of the medical image is 3 mm.

13. The method of claim 1, wherein automatically generating the region of interest in the medical image for application of tumor treating fields to the subject comprises:
providing for display a user-adjustable value for a proximal boundary zone (PBZ) for expanding a segmented area in the medical image to obtain an expanded segmented area in the medical image.

14. The method of claim 1, wherein the minimum number of voxels of the medical image for segmentation is 10 voxels.

15. The method of claim 1, wherein if the minimum number of voxels of the medical image for segmentation is not satisfied, a warning is provided to a user.

16. The method of claim 1, further comprising:
generating a plurality of transducer layouts for application of tumor treating fields to the subject based on the automatically generated region of interest in the medical image.

17. The method of claim 1, further comprising:
creating a three-dimensional model of the subject based on the medical image, the three-dimensional model of the subject including the automatically generated region of interest;
generating a plurality of transducer layouts for application of tumor treating fields to the subject based on the three-dimensional model of the subject;
selecting at least two of the plurality of transducer layouts as recommended transducer layouts;
presenting the recommended transducer layouts;
receiving a user selection of at least one recommended transducer layout; and
providing a report for the at least one selected recommended transducer layout.

18. The method of claim 1, wherein the medical image comprises at least one of a computed tomography (CT) medical image, a magnetic resonance imaging (MRI) medical image, or a positron emission tomography (PET) medical image.

19. A non-transitory processor readable medium containing a set of instructions thereon for treatment planning for administering tumor treating fields to a subject, wherein when executed by a processor, the instructions cause the processor to perform a method comprising:
presenting on a display a slice through a medical image of the subject, wherein the medical image comprises voxels; and
determining a segmentation of a minimum number of voxels of at least one tissue type in the slice of the medical image;
receiving a user selection to automatically generate a region of interest (ROI) in the medical image for application of tumor treating fields to the subject; and
automatically generating the region of interest in the medical image for application of tumor treating fields to the subject based on the segmentation of the minimum number of voxels of at least one tissue type in the slice of the medical image.

20. An apparatus for treatment planning for administering tumor treating fields to a subject, the apparatus comprising: one or more processors; and memory accessible by the one or more processors, the memory storing instructions that when executed by the one or more processors, cause the apparatus to perform a method comprising:
presenting on a display a slice through a medical image of the subject, wherein the medical image comprises voxels; and
determining a segmentation of a minimum number of voxels of at least one tissue type in the slice of the medical image;
receiving a user selection to automatically generate a region of interest (ROI) in the medical image for application of tumor treating fields to the subject; and
automatically generating the region of interest in the medical image for application of tumor treating fields to the subject based on the segmentation of the minimum number of voxels of at least one tissue type in the slice of the medical image.
